(19) United States
(12) Patent Application Publication
(10) Pub. No.: US 2014/0142686 A1
(43) Pub. Date: May 22, 2014

(54) BIODEGRADABLE STENT FORMED WITH POLYMER-BIOCERAMIC NANOPARTICLE COMPOSITE AND METHOD OF MAKING THE SAME

(71) Applicant: Tim Wu, Shrewsbury, MA (US)

(72) Inventor: Tim Wu, Shrewsbury, MA (US)

(73) Assignee: Tim Wu, Shrewsbury, MA (US)

(21) Appl. No.: 13/684,226

(22) Filed: Nov. 22, 2012

(51) Int. Cl. A61F 2/82

(52) U.S. Cl. CPC A61F 2/82
USPC 623/1.38; 264/117

(57) ABSTRACT

The present invention relates to biodegradable medical devices such as stents manufactured from biodegradable polymeric-bioceramic nanoparticle composites. The invented medical devices include at least one bioceramic nanoparticle dispersed in at least one biodegradable polymer, wherein the said biodegradable polymers include biodegradable polyesters. The device and methods to disperse one or more bioceramic nanoparticle, wherein the said bioceramic nanoparticle include, but are not limited to, amorphous calcium phosphate (ACP), dicalcium phosphate (DCP), tricalcium phosphate (TCP), pentacalcium hydroxyl Apatite(HAp), tetracalcium phosphate monoxide(TTCP) and combinations or analogues thereof. Other embodiments include methods of fabricating biodegradable stent with said polymeric-nanoparticle composites.

### CROSS-REFERENCE TO RELATED APPLICATIONS

This application is a continuation-in-part of the U.S. patent application Ser. No. 13/014,750, filed on Jan. 27, 2011, which is the continuation-in-part of the U.S. patent application Ser. No. 11/843,528, filed on Aug. 22, 2007, which claims the benefit of U.S. provisional patent application No. 60/823,168, filed on Aug. 22. 2006. This application is also a continuation-in-part of the U.S. patent application Ser. No. 12/209,104, filed on Sep. 11, 2008, which claims the benefit of U.S. provisional patent application No. 60/578,219, filed on Jun. 8, 2004. This application also claims the benefit of the U.S. provisional application No. 61/368,833, filed on Jul. 29, 2010 and U.S. provisional patent application No. 61/427,141 tiled on Dec. 24, 2010. The disclosures of all of which are hereby incorporated by reference in their entireties.

### FIELD OF THE INVENTION

The present invention relates to a biodegradable stent comprising at least one bioceramic nanoparticle encapsulated inside at least one biodegradable polymer wherein the encapsulated bioceramic nanoparticle would improve the said biodegradable polymer's biocompatibility, modify the said biodegradable polymer's degradation rate and enhance the said biodegradable polymer's mechanic properties.

The present invention encompasses the discovery that at least one bioceramic nanoparticle can be encapsulated into at least one biocompatible polymer through extrusion or injection molding process to form a tubular structure for subsequent biodegradable stent fabrication. The formed tube has improved biocompatibility, reinforced mechanic property and modified degradation rate.

The present invention further provides the methods of fabricating the polymer-bioceramic composite made implantable biodegradable medical device such as biodegradable stent that effectively controls sustained-release of the therapeutic agent. The present invention also encompasses the finding that medical devices made from the invented polymeric-nanoparticle composite have surprisingly improved biocompatibility, modified biodegradation and increased device's radial strength. The present invention therefore provides, among other things, a nanoparticle encapsulated implantable medical device comprising a bioceramic nanoparticle, encapsulated in at least one biocompatible and biodegradable polymer. The present invention further provides medical devices encapsulated with at least one bioceramic nanoparticle according to the invention and methods of making the same.

In one aspect, the present invention related to a nanoparticle-enhanced implantable medical device, comprising at least one nanoparticle encapsulated inside at least one biocompatible polymer, wherein the nanoparticle are functioned to improve the device's biocompatibility modify the device's degradation rate and increase the device's mechanic properties.

In some embodiments, suitable bioceramic nanoparticle is selected from the groups consisting of calcium phosphate family including, but are not limited to, amorphous calcium phosphate (ACP), dicalcium phosphate (DCP), tricalcium phosphate (TCP), pentacalcium hydroxyl Apatite(HAp), tetracalcium phosphate monoxide(TTCP) and combinations or analogues thereof.

In some embodiments, polymers suitable for the present invention contains a biodegradable polymer. In some embodiments, the biodegradable polymer is a polyester polymer. In some embodiments, suitable polyester polymer include, but are not limited to, poly (D,L-lactide-co-glycolide) (PLGA), polylactide (PLA), poly(L-lactide) (PLLA), poly(D,L-lactide (PDLA), polyglycolides (PGA), poly(D,L-glycolide) (PLG), and combinations thereof.

In another aspect, the present invention provides methods for fabricating nanoparticle encapsulated implantable medical device, more specifically, a biodegradable stent, including polymeric-nanoparticle composite compounding, polymeric-nanoparticle composite tube forming, polymeric and nanoparticle molecular orientation, stent laser cutting etc. In some embodiments, the compoundable polymer and nanoparticle are crystallized by various nanotechnologies and the nanoparticle-containing tube is then extruded through an extruder or injection molding with the polymeric-nanoparticle composite at the temperature of equal or above polymer melting point. In one embodiment, the nanoparticle-sized polymer and nanoparticle are premixed before extrusion or molding and be extruded to solidified tubular structure through extruder under the temperature above the polymer's melting point.

In some embodiment, the formed tubes are further deformed radially and axially to orientate both the polymer and nanoparticle molecule direction with the blow molding technology to increase the tube's mechanic strength and particle's crystallinity. The deformed tubes are then subjected to laser cutting which is a know art according to the stent design pattern.

### BACKGROUND OF THE INVENTION

Coronary Artery Disease (CAD) has been the number one killer in the United States since. 1900 and still remains the most common cause of death in the Western world despite therapeutic advances. Drug-Eluting Stent (DES) is currently the major therapy for CAD treatment. DES not only increases procedural success rates, but also increases the safety of procedures by decreasing the need for emergency coronary artery bypass graft surgery (CABG). As a result, stents are currently utilized in over 85% of the two million Percutaneous Coronary Intervention procedures (PCIs) in the US. The total direct cost for these life-saving procedures is over $2 billion annually. Despite the prevalent use of DES, there are significant drawbacks, including the need for costly, long-term anti-platelet therapy, as well as the metal artifact remaining in the vessel. Coronary stents are only required to provide scaffolding for up to six months following the procedure, however, since the stent remains in the vessel, potential long term complications may arise. In addition, the remaining metal scaffolding precludes the vessel from returning to its natural state and prevents true endothelial repair and arterial remodeling. Those drawbacks had caused two major issues in current DESs including in-stent restenosis and late stage thrombosis.

In-Stent Restenosis (ISR) is the re-narrowing of an opened artery after stenting due primarily to the proliferative response of the intima, a layer of cells that line the lumen of the vessel, composed of connective tissue and smooth muscle cells (SMC). ISR has been the biggest problem in PCI until the recently successful development of DESs. Initially, the restenosis rate is as high as over 50% within six months post balloon dilation. Stenting lowers this number to 20-30%. DESs can significantly reduce the rate of restenosis to <10%. However, ISR in patients with high risk such as small vessels, diabetes, and long diffusion diseased arteries still remains unacceptably high (30%-60% in bare metal stents and 6%-18% in DESs).

Thrombosis: in spite of restenosis remaining a clinical problem in approximately 10% with DES implantation, it can often be successfully treated with repeated DES implantation. The greatest concern, however, has been of stent thrombosis which is associated with a high rate of myocardial infarction and death. The rate of early stent thrombosis (less than 30 days following implantation) appears similar in both bare metal stents (BMS) and DESs. However, late stent thrombosis (LST) has been increasingly reported beyond 12 months following DES implantation, with the greatest risk occurring as a result of premature discontinuation of antiplatelet therapy. Although the precise mechanism of late stage stent thrombosis is unknown, it is generally believed that the combination of delayed endothelialization due to antiproliferative therapy and persistence of the nonerodable polymer contribute to the hypersensitivity reaction, possibly with some residual active drug that may not be eluted.

Therefore, the challenges faced by emerging technologies are to reduce restenosis in high-risk lesions without compromising healing in order to avoid late thrombotic complications, and to improve system deliverability in order to allow the devices to treat more complex patients. Currently, a number of strategies are being utilized to achieve these goals, through the development of novel stent platforms, coating with biodegradable polymer or move away from polymers, and with new generations and/or combinations of biological agents that both inhibit proliferation and promote endothelialization. With the recent positive data from Abbott's ABSORB trial, clinical consensus is building that fully biodegradable stents (BDS) represent the next generation in DES.

Bioabsorbable and biodegradable materials for manufacturing temporary stents present a number of advantages. The conventional bioabsorbable or bioresorbable materials of the stents are selected to absorb or degrade over time to allow for subsequent interventional procedures such as restenting of the original site if there is restenosis and insertion of a vascular graft. Further, bioabsorbable and biodegradable stents allow for vascular remodeling, which is not possible with metal stents that tethers the arterial wall to a fixed geometry. In addition to the advantages of not having to surgically remove such stents, bioabsorbable and biodegradable materials tend to have excellent biocompatibility characteristics, especially in comparison to most conventionally used biocompatible metals. Another advantage of bioabsorbable and biodegradable stents is that the mechanical properties can be designed to

TABLE 1
Potential Benefits of Biodegradable Stent

May limit late-stent thrombosis
Allows late favorable positive remodeling
May reduce long-term dual antiplatelet therapy
Has Larger drug-loading capacity
Addresses patient's concerns about permanent implants
Faciltates noninvasive diagnosis imaging(MR/CT)
Surgical option not restricted
Easier repeat revascularization

substantially eliminate or reduce the stiffness and hardness that is often associated with metal stents, which can contribute to the propensity of a stent to damage a vessel or lumen. Examples of novel biodegradable stents include those found in U.S. Pat. No. 5,957,975, and U.S. application Ser. No. 10/508,739, which is herein incorporated by reference in its entirety. Table 1 summarizes the potential advantages of fully BDS over nonbiodegradable stent.

Biodegradable polyester polymer including polylactides (PLA), polyglycolides (PGA) and their copolymer PLGA are the major polymers currently used in making BDS. The advantage of polyester polymer is that its degradation products are ultimately converted to water and carbon dioxide through the action of enzymes in the tricarboxylic acid cycle and are excreted via the respiratory system. However, there are several major issues existed in current biodegradable stent including: 1) the significant inflammatory response of the vessel wall caused by accumulated acidic products during polymer degradation, leads to worse restenosis than that is caused by a metal stent. 2) The lack of sufficient radial strength to support collapsed vessels and to prevent it from recoiling. Other limitations in polymer alone stents include: radiolucent which may impair accurate positioning, and limited mechanical performance which requires thick struts that impede their profile and delivery capabilities.

Therefore, the present invention provides a biodegradable stent system made from a biodegradable polymer-bioceramic nanoparticle composite with reinforced mechanic property, improved biocompatibility, and adjustable degradation rate.

### SUMMARY OF THE INVENTION

In one aspect, the present invention provide a bioabsorbable stent made from a polymer-bioceramic nanoparticle composite, wherein at least one bioceramic nanoparticle were encapsulated inside at least one biodegradable polymer, more specifically, biodegradable polyester polymer. The bioceramic nanoparticle encapsulated into said biodegradable polymer include, but are not limited to, amorphous calcium phosphate (ACP), dicalcium phosphate (DCP), tricalcium phosphate (TCP), pentacalcium hydroxyl Apatite(HAp), tetracalcium phosphate monoxide(TTCP) and combinations or analogues thereof.

In other aspect, the present invention include a bioabsorbable medical device made from a polymer-nanoparticle composite wherein at least one of nanoparticle were encapsulated inside at least one biodegradable polymer, more specifically, biodegradable polyester polymer. The bioceramic nanoparticle encapsulated into the said biodegradable polymer include, but are not limited to, amorphous calcium phosphate (ACP), dicalcium phosphate (DCP), tricalcium phosphate (TCP), pentacalcium hydroxyl Apatite(HAp), tetracalcium phosphate monoxide(TTCP) and combinations or analogues thereof.

In another aspect, the present invention includes a method of fabricating an implantable medical device with the polymer-nanoparticle composite. The method includes the operations of nanoparticle and polymeric composition compounding, polymer-nanoparticle composite tube forming, polymeric and nanoparticle molecular orientation, stent laser cutting etc.

In another aspect, the present invention includes a method of fabricating a biodegradable stent with bioceramic nanoparticle-containing polymeric composition. The method includes the following processing operations: nanoparticle and polymer pre-crystallization and polymeric composition compounding with various nanotechnologies, nanoparticle-containing polymeric composition tube forming, polymeric and nanoparticle molecular orientation, stent laser cutting etc. The nanoparticle encapsulated inside the polymer include, but are not limited to, amorphous calcium phosphate (ACP), dicalcium phosphate (DCP), tricalcium phosphate (TCP), pentacalcium hydroxyl Apatite(HAp), tetracalcium phosphate monoxide(TTCP) and combinations or analogues thereof.

Preferably, the biodegradable stent made from invented polymeric composite has at least 10% improvement of stent's biocompatibility, expedited material degradation and mechanic property increase than that made from no nanoparticle encapsulated polymer only. More preferably, 50% improvement of stent's biocompatibility, expedited material degradation duration and mechanic property increase than that made from no nanoparticle encapsulated polymer only. Most preferably, at least 90%, 95%, or 98% of improvement of stent's biocompatibility, expedited material degradation duration and mechanic property increase than that made from no nanoparticle encapsulated polymer only.

### BRIEF DESCRIPTION OF THE DRAWING

FIG. 1, Illustration of an exemplary biodegradable drug-eluting stent (PowerStent® Absorb) of the invention. Upper: PowerStent® Absorb expanded at 3 mm; Lower: Expanded PowerStent® Absorb after withdrawing the delivery balloon.

FIG. 2 SEM micrographs of cross sections from PLLA and PLLA/ACP tubes before and after 6 weeks hydrolic degradation (A-D) and of the PowerStent® Absorb stent surface after laser cutting (E-F). A: PLLA tube before; B: PLLA tube 6 weeks; C: PLLA/ACP tube before; and D: PLLA/ACP tube at 6 weeks. Note the uniform dispersion of ACP in PLLA (C) and the significantly structural deference between PLLA and PLLA/ACP tube at 6 weeks degradation (B VS. D). E: ×140 magnification of a precision laser cut PowerStent® Absorb stent; F: ×1000 magnification of cross sectional surface (red boxed area in A) revealing the nanopore surface structure of PLLA/ACP composite strut; G: ×7500 magnification of stent surface (yellow boxed area in A) revealing the smooth extruded stent surface; H: ×5000 magnification of laser cut stent revealing ACP nanoparticles uniformly dispersed in PLLA polymer.

FIG. 3, Exemplary biocompatibility (morphometric) comparison between stents made from PLLA and PLLA-ACP composite at one month post implantation in pig coronary arteries. Please note that PLLA stent has significantly greater percentage of restenosis, inflammatory scores, and stent recoil rates than PLLA/ACP stent at one month post implantation (P<0.05).

FIG. 4, Exemplary angiographic and histopathological comparisons of PLLA and PLLA/ACP (PowerStent® Absorb) stents in porcine coronary artery at one month post implantation. Left panel (A-F): PLLA stent; Right panel (A1-F1): PLLA/ACP stent. A & A1: Angiographs at deployment; B & B1: Angiographs after 1 month. Please note the mottled texture of the inner arterial wall in the representative PLLA sample after 1 month (B, boxed area) in contrast to the smooth arterial wall in A, A₁ and B₁. C & C1: Gross pathological observations at one month follow-up. Please note the significant inflammatory response in C (boxed area) compared to the relatively minor inflammation in C₁ exposed once after adipose was dissected away). D & D1: Crossing section of C & C1 (4×). Note the massive neointima formation in D (yellow arrow) compared to the thin layer of endothelial cells covering stent strut in D₁ (yellow arrow) and the difference of stent recoil between the two treatment groups (yellow-dash line circulated area in D and D₁). E & E1: High magnification (20×) of blue boxed area in D and D₁. Please note the significant less number of inflammatory cell infiltration in PLLA/ACP group (E₁) than that in PLLA group (E).

FIG. 5, Exemplary case-by-case histopathoogical comparisons of PLLA and. PLLA/ACP (PowerStent® Absorb) stented coronary arteries at one month post deployment. Left: PLLA/ACP stents at 4× and 20×; Right: PLLA stents at 4× and 20×. Please note that 4 of 5 stented arteries in PLLA group have massive neointima formation (stent#1-4) compared to none in the PowerStent® Absorb group. All samples in PowerStent® Absorb group showed struts covered with thin layer of cells. Please also note that 100% of the PLLA stented arteries show evidence of stent recoil, with struts separated from arterial wall (stent recoil), vs. none from the PowerStent® Absorb group.

FIG. 6, Exemplary mechanic property comparison between PLLA and PLLA/ACP tubes before and after 6 weeks hydrolic degradation. The radial strength of PLLA/ACP was significantly greater than for the PLLA tubes before and after 6 weeks degradation (N=12 each, P<0.01 at each time point).

FIG. 7, Summarize the changes in physical properties of Poly-L-lactide (PLLA) versus PLLA/ACP tubes before and and 6 months hydrolic degradation in-vitro.

### DEFINITIONS

Agent: As used herein, the term “agent” refers to any substance that can be delivered to a tissue, cell, vessel, or subcellular locale. In some embodiments, the agent to be delivered is a biologically active agent (bioactive agent), i.e., it has activity in a biological system and/or organism. For instance, a substance that, when introduced to an organism, has a biological effect on that organism, is considered to be biologically active or bioactive. In some embodiments, an agent to be delivered is an agent that inhibit, reduce or delay cell proliferation.

Polymer: As used herein, the term “polymer” refers to any long-chain molecules containing small repeating units.

Therapeutic agent: As used herein, the phrase “therapeutic agent” refers to any agent that, when administered to a subject, has a therapeutic effect and/or elicits a desired biological and/or pharmacological effect.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition (e.g., hyperproliferation such as restenosis). Treatment may be administered to a subject who does not exhibit signs of a disease and/or exhibits only early signs of the disease for the purpose of decreasing the risk of developing pathology associated with the disease.

Stenosis and Restenosis: As used herein, the term “Stenosis” refers to a narrowing or constriction of the diameter of a bodily passage or orifice. In stent related treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Nanoparticle: The term “nano-particles” or “micro-particles” is used throughout the present invention to denote carrier structures that are biocompatible and have sufficient resistance to chemical and/or physical destruction by the environment of use such that a sufficient amount of the nano-particles and/or micro-particles remain substantially intact after injection into a target site in the arterial wall. Typically, the nano-particles of the present invention have sizes ranging from about 1 nm to about 1000 nm, with sizes from about 100 nm to about 500 nm being more preferred. The micro-particles of the present invention have sizes ranging from about 1 .mu.m to about 1000 .mu.m, with sizes from about 10 .mu.m to about 200 .mu.m being more preferred.

Stress: as used herein, the term “stress” refers to force per unit area, as in the force acting through a small area within a plane. Stress can be divided into components, normal and parallel to the plane, called normal stress and shear stress, respectively. True stress denotes the stress where force and area are measured at the same time. Conventional stress, as applied to tension and compression tests, is force divided by the original gauge length.

Strength: as used herein, the term “strength” refers to the maximum stress along an axis which a material will withstand prior to fracture. The ultimate strength is calculated from the maximum load applied during the test divided by the original cross-sectional area.

Modulus: as used herein, the term “Modulus” is defined as the ratio of a component of stress or force per unit area applied to a material divided by the strain along an axis of applied force that results from the applied force. For example, a material has both a tensile and a compressive modulus. A material with a relatively high modulus tends to be stiff or rigid. Conversely, a material with a relatively low modulus tends to be flexible. The modulus of a material depends on the molecular composition and structure, temperature of the material, amount of deformation, and the strain rate or rate of deformation.

Strain: as used herein, the term “strain” refers to the amount of elongation or compression that occurs in a material at a given stress or load.

Elongation: as used herein, the term “elongation” may be defined as the increase in length in a material which occurs when subjected to stress. It is typically expressed as a percentage of the original length. Elongation to Break is the strain on a sample when it breaks. It is usually is expressed as a percent.

Toughness: toughness is the amount of energy absorbed prior to fracture, or equivalently, the amount of work required to fracture a material. One measure of toughness is the area under a stress-strain curve from zero strain to the strain at fracture. The stress is proportional to the tensile force on the material and the strain is proportional to its length. The area under the curve then is proportional to the integral of the force over the distance the polymer stretches before breaking. This integral is the work (energy) required to break the sample. The toughness is a measure of the energy a sample can absorb before it breaks. There is a difference between toughness and strength. A material that is strong not tough is said to be brittle. Brittle substances are strong, but cannot deform very much before breaking.

Solvent: the solvent is defined as a substance capable of dissolving or dispersing one or more other substances or capable of at least partially dissolving or dispersing the substance(s) to form a uniformly dispersed solution at the molecular- or ionic-size level at a selected temperature and pressure. The solvent should be capable of dissolving at least 0.1 mg of the polymer in 1 ml of the solvent, and more narrowly 0.5 mg in 1 ml at the selected temperature and pressure, for example, ambient temperature and ambient pressure.

Composite: A “composite” refers generally to a material in which two or more distinct, structurally complementary substances combine to produce structural or functional properties not present in any individual components.

### DETAILED DESCRIPTION OF THE INVENTION

Various embodiments of the present invention include a stent having a stent body formed at least in part from a polymeric matrix composite, the composite including bioceramic particles dispersed within a biodegradable polymer. The bioceramic particles can also be bioabsorbable. In some embodiments, the dispersed bioceramic particles modify the in-vivo degradation rate of polymeric matrix, and thus, of the composite and the stent body. In some embodiment, the bioceramic particles enhance the mechanical properties of the composite, and thus, the stent body. In some other embodiment, the bioceramic particles improve the biocompatibility of the composite, and thus, the stent body by neutralizing, the acidic product generated from polymer degradation. The various publication from inventors showed that the degradation rate, the mechanical properties and the biocompatibility of a stent made by a bioceramic-polymeric matrix in the present invention are adjustable due to the bioceramic particles.

Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels or restenosis in an opened blood vessel or heart valve.

The treatment of a diseased site or lesion with a stent involves both delivery and deployment of the stent. “Delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self-expanding stent, the stent may be secured to the catheter via a constraining member such as a retractable sheath or a sock. When the stent is in a desired bodily location, the sheath may be withdrawn which allows the stent to self-expand.

Stents suitable for the present invention include any stent for medical purposes, which are known to the skilled artisans. Exemplary stents include, but are not limited to, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,371 issued to Pinchasik et al.

Suitable stents for the present invention are biodegradable non-metal stent. Exemplary biocompatible non-metal stents include, but not limited to, stents made from carbon, carbon fiber, cellulose acetate, cellulose nitrate, silicone, polyethylene teraptithalate, polyurethane, polyamide, polyester, polyorthoester, polyanhydride, polyether sulfone, polycarbonate, polypropylene, polyethylene, polytetrafluoroethylene, polylactic acid, polyglycolic acid, a polyanhydride, polycaprolactone, polyhydroxybutyrate, or combinations thereof. Other polymers suitable for non-metal stents are shape-memory polymers, as described for example by Froix, U.S. Pat. No. 5,163,952, which is incorporated by reference herein. Stents formed of shape-memory polymers, which include metharylate-containing and acrylate-containing polymers, readily expand to assume a memory condition to expand and press against the lumen walls of a target vessel, as described by Phan, U.S. Pat. No. 5,603,722, which is incorporated by reference in its entirety.

In one aspect, the suitable biodegradable polymer for the present invention include any polymers that are biologically inert and not induce further inflammation (e.g., biocompatible and avoids irritation to body tissue). In some embodiments, the suitable polymers in the present invention are polyester biodegradable polymers. Exemplary biodegradable polymers include, but are not limited to poly(L-lactide), poly(D,L-lactide, poly(L-lactide-co-D,L-lactide), poly(L-lactide-co-glycolide), poly(D,L-lactide-co-glycolide), poly(L-lactide-co-caprolactone), poly(glycolide-co-caprolactone), poly(D,L-lactide-co-caprolactone) and blends of the aforementioned. PLA and PGA are desirable for medical applications because they have lactic acid and glycolic acid as their degradation products, respectively. These natural metabolites are ultimately converted to water and carbon dioxide through the action of enzymes in the tricarboxylic acid cycle and are excreted via the respiratory system. In addition, PGA is also partly broken down through the activity of esterases and excreted in the urine. Along with its superior hydrophobicity, PLA is more resistant to hydrolytic attack than PGA, making an increase of the PLA:PGA ratio in a PLGA copolymer result in delayed degradability.

In one aspect, the bioceramic nanoparticle in the present invention include, but are not limited to, any ceramic material that is compatible with the human body. More generally, include any type of compatible inorganic material or inorganic/organic, hybrid material. Bioceramic materials can include, but are not limited to, alumina, zirconia, apatites, calcium phosphates, silica based glasses, or glass ceramics, and pyrolytic carbons. Bioceramic materials can be bioabsorbable and/or active. A bioceramic is active if it actively takes part in physiological processes. A bioceramic material can also be “inert,” meaning that the material does not absorb or degrade under physiological conditions of the human body and does not actively take part in physiological processes.

Exemplary bioceramic nanoparticle are apatites and other calcium phosphates, include, but are not limited to hydroxyapatite (Ca.sub.10(PO.sub.4).sub.6(OH).sub.2), floroapatite (Ca.sub.10(PO.sub.4).sub.6F.sub.2), carbonate apatide (Ca.sub.10(PO.sub.4).sub,6CO.sub.3), tricalcium phosphate (Ca.sub.3(PO.sub.4).sub.2), octacalcium phosphate (Ca.sub.8H.sub.2 (PO.sub.4)6-5H.sub.2O), octacalcium phosphate (Ca.sub.8H.sub.2 (PO.sub.4)6-5H.sub.2O), calcium pyrophosphate (Ca.sub.2P.sub.2O.sub.7-2H.sub.2O), tetracalcium phosphate (Ca.sub.4P.sub.2O.sub.9), and dicalcium phosphate dehydrate (CaHPO.sub.4-2H.sub.2O).

The term bioceramics can also include bioactive glasses that are bioactive glass ceramics composed of compounds such as SiO.sub.2, Na.sub2O, CaO, and P.sub.2O.sub.5. For example, a commercially available bioactive glass, Bioglass®, is derived from certain compositions of SiO.sub.2-Na2O—K.sub.2O—CaO—MgO—P.sub.2O.sub.5 systems. Some commercially available bioactive glasses include, but are not limited to:

45S5: 46.1 mol % SiO.sub.2, 26.9 mol % CaO, 24.4 mol % Na.sub.2O and 2.5 mol % P.sub.2O.sub.5;

58S: 60 mol. % SiO2, 36 mol % CaO, and 4 mol % P.sub.2O.sub.5; and

S70C30: 70 mol % SiO2, 30 mol % CaO.

Another commercially available glass ceramic is A/W.

Bioceramic particles can be partially or completely made from a biodegradable, bioabsorbable, or biostable ceramic. Examples of bioabsorbable bioceramics include hydroxyapatite, various types of bioglass materials, tetracalcium phosphate, amorphous calcium phosphate, alpha-tricalcium phosphate, and beta-tricalcium phosphate. Biostable bioceramics include alumina and zirconia.

In some embodiments, the concentration of bioceramic particles in the composite can be adjusted to obtain a selected degradation rate and degradation time of an biodegradable stent. Adjusting the concentration of bioceramic particles can change the degradation rate due to both the change in pH level and the amount or mass of the polymer matrix exposed to the degradation products. Exemplary embodiments of a composite of stent can have a concentration of bioceramic particles ranges from about 99:1 to 1:99 (e.g., 10:90, 20:80, 30:70, 40:60, 50:50, 60:40, 70:30, 80:20, 90:10).

Exemplary bioceramic agent that may be used in the current invention include, but not limited to, amorphous calcium phosphate (ACP), dicalcium phosphate (DCP), tricalcium phosphate (TCP), pentacalcium hydroxyl Apatite(HAp), tetracalcium phosphate monoxide(TTCP) and combinations or analogues thereof.

For example, ACP is an important intermediate product for in vitro and in vivo apatite formation with high solubility and better biodegradability. It was mainly used in the form of particles or powders, as an inorganic component incorporated into biopolymers, to adjust the mechanical properties, biodegradability, and bioactivity of the resulting composites. Based on the similarity of ACP to the inorganic component of the bone, ACP is particular useful as a bioactive additive in medical devices to improve remineralization. Based on its solubility, coatings containing ACP may release ions into aqueous media, forming a favorable super saturation level of Ca2+ and PO43− ions for the formation of apatite. The ion release may neutralize the acidity resulted from polymer biodegradation, retarding, bioresorptive rate and eliminating inflammation occurrence.

In one aspect, biodegradable stent made from polymeric-nanoparticle composite may also include a therapeutic or other specific beneficial agent that is released into the vessel for treatment thereof as stent biodegrades. A wide range of therapeutic agents can be used, with the pharmaceutically effective amount being readily determined by those of ordinary skill in the art and ultimately depending, for example, upon the condition to be treated, the nature of the therapeutic agent itself, the tissue into which the dosage form is introduced, and so forth. For example, the therapeutic agents may include one or more of the following: anti-thrombotic agents, anti-proliferative agents, anti-inflammatory agents, anti-migratory agents, agents affecting extracellular matrix production and organization, antineoplastic agents, antimitotic agents, anesthetic agents, anti-coagulants, vascular cell growth promoters, vascular cell growth inhibitors, cholesterol-lowering agents, vasodilating agents, and agents that interfere with endogenous vasoactive mechanisms. The therapeutic agents may be disposed within the filament or attached to the surface of the filament as a coating. The detail of the suitable therapeutic which can be used and the methods of the encapsulating those therapeutic agents into the biodegradable polymer has been fully disclosed in prior patent application Ser. No. 12/209,104, filed on Sep. 11, 2008, and provisional patent application No. 61/427,141 filed on Dec. 24, 2010.

In some embodiment, the mechanic property of invented stent is increased by adding bioceramic nanoparticle in to the polymer. The stent must be able to satisfy a number of mechanical requirements. First, the stent must be capable of withstanding the structural loads, namely radial compressive forces, imposed on the stent as it supports the walls of a vessel. Therefore, a stent must possess adequate radial strength. Radial strength, which is the ability of a stent to resist radial compressive forces, is due to strength and rigidity around a circumferential direction of the stent. Radial strength and rigidity, therefore, may also be described as, hoop or circumferential strength and rigidity.

Once expanded, the stent must adequately maintain its size and shape throughout its service life despite the various forces that may come to bear on it, including the cyclic loading induced by the beating heart. For example, a radially directed force may tend to cause a stent to recoil inward. Generally, it is desirable to minimize recoil. In addition, the stent must possess sufficient flexibility to allow for crimping, expansion, and cyclic loading. Longitudinal flexibility is important to allow the stent to be maneuvered through a tortuous vascular path and to enable it to conform to a deployment site that may not be linear or may be subject to flexure. Finally, the stent must be biocompatible so as not to trigger any adverse vascular responses.

The structure of a stent is typically composed of scaffolding that includes a pattern or network of interconnecting structural elements often referred to in the art as struts or bar arms. The scaffolding can be formed from wires, tubes, or sheets of material rolled into a cylindrical shape. The scaffolding is designed so that the stent can he radially compressed (to allow crimping) and radially expanded (to allow deployment). A conventional stent is allowed to expand and contract through movement of individual structural elements of a pattern with respect to each other.

In some embodiment, the biocompatibility of the stent in the present invention is improved by adding the bioceramic nanoparticle to the biodegradable. Biocompatibility is related to the behavior of biomaterials in various contexts. The term may refer to specific properties of a material without specifying, where or how the material is used (for example, that it elicits little or no immune response in a given organism, or is able to integrate with a particular cell type or tissue), or to more empirical clinical success of a whole device in which the material or materials feature.

As well known, polyester biodegradable material is a widely used material in making biodegradable products in the area of bone tissue regeneration, cardiovascular devices, drug delivery vehicles etc. as their degradation products are ultimately converted to water and carbon dioxide through the action of enzymes in the tricarboxylic acid cycle and are excreted via the respiratory system. However, polyester biodegradable polymer also generate acidic by-product during degradation process which can cause stented arterial tissue inflammation. The adding of bioceramic nanoparticle can neutralize those acidic by-products, and therefore improve the stent biocompatibility.

Also, in some certain embodiment, the degradation rate of the polymer-nanoparticle composite can be modified by adjusting the pH local to the stent. Local regions refer to regions within the composite, on the surface of the composite, or adjacent to the composite. The local pH is adjusted by the degradation products of bioceramic particles incorporated within or on the stent. The local pH is adjusted without administering alkalizing or acidic substances systemically to the patient.

Stents have typically been constructed of relatively inert metals in order to ensure their longevity. Degradable or erodible stent structures have more recently been devised in an effort to provide support for only a limited period of time. In general, the support or patency provided by a stent for the treatment of a stenosis is required only for a limited period of time. For example, a preferred or required treatment time by a stent may be less than eighteen months, less than a year, between three and twelve months, or more narrowly, between four and eight months. Thus the degradation rate of stent need to be adjusted to tailed according to the clinical need.

Various environmental factors can influence the rate of degradation including, but are not limited to, hydrogen-ion concentration (pH) in the solution, influence of oxygen in solution adjacent to the polymer, specific nature and concentration of other ions in solution, rate of flow of the solution in contact with the polymer, temperature, and cyclic stress (degradtion fatigue). In particular, a change in pH can influence degradation by affecting reaction kinetics of the degradation reactions and by affecting the passivation or ability to form a protective layer. With regard to passivation, the ability to form a protective layer can depend on the solubility of protective layer materials. The solubility of these materials can depend on the pH of the degradation environment.

In some embodiment of the invention, various sizes of the bioceramic particles may be used in the composite. For example, the bioceramic particles can include, but are not limited to, nanoparticles and/or micro particles. A nanoparticle refers to a particle with a characteristic length (e.g., diameter) in the range of about 1-1,000 nm, or more narrowly in the range of 1-100 nm. A microparticle refers to a particle with a characteristic length in the range of greater than 1,000 nm and less than about 10 micrometers. Additionally, bioceramic particles can be of various shapes, including but not limited to, spheres and fibers.

In some embodiment, the size of the bioceramic particles can be adjusted to tailor the mechanic strength and degradation rate. Bioceramic nanoparticles may be more effective in modifying the erosion rate of the polymer matrix than microparticles. Since nanoparticles have a larger surface to volume ratio than larger particles, they are expected to provide a greater and more uniform exposure to degradation products than larger particles.

In some embodiments, the concentration of bioceramic particles in the composite can be adjusted to obtain a selected degradation rate, mechanic strength and biocompatibility improvement. Adjusting the concentration of bioceramic particles can change the degradation rate. Mechanic strength and biocompatibility due to both the changes in pH level and the amount or mass of the polymeric matrix exposed to the degradation products. Exemplary embodiments of a composite of stent can have a concentration of bioceramic particles ranges from about 99:1 to 1:99 (e.g., 10:90, 20:80, 30:70, 40:60, 50:50, 60:40, 70:30, 80:20, 90:10).

In various embodiments of the invention, the dispersed bioceramic particles can act as a reinforcing material to enhance the mechanical properties of the matrix such as toughness, stiffness, and strength. In general, the higher the fracture toughness, the more resistant a material is to the propagation of cracks. Certain regions of an implantable medical device, such as a stent, experience a high degree of stress and strain when the device is under stress during use. For example, when a stent is crimped and deployed, curved or bending regions can have highly concentrated strain which can lead to fracture. The bioceramic particles can increase fracture toughness by reducing the concentration of strain by dispersing the strain over a larger volume of the material. Particles can absorb energy due to applied stress and disperse energy about a larger volume in the bioceramic-polymer matrix composite.

Therefore, rather than being highly concentrated, the stress and strain in a stent fabricated from a bioceramic-polymer matrix composite is divided into many small interactions involving numerous individual particles. When a crack is initiated in the material and starts traveling through the composite, the crack breaks up into finer and finer cracks due to interaction with the particles. Thus, the particles tend to dissipate the energy imparted to the stent by the applied stress.

Additionally, the use of nanoparticles may be particularly advantageous in improving mechanical properties. For a give weight ratio of particles to polymer matrix, as the size of the particles decreases the number of particles dispersed throughout the stent per unit volume also increases. Thus, the number of particles available to disperse the energy of applied stress to the stent increases. Therefore, it is expected that a composite with nanoparticles will result in a more uniform and greater enhancement of mechanical properties.

Additionally, the dispersed bioceramic particles can increase the strength of the composite. As indicated above, a stent requires a high radial strength in order to provide effective support to a vessel. A composite having dispersed bioceramic particles with may have a higher strength than the polymer only. It is believed that the bioceramic particles will enhance the strength and toughness during all or a portion of the time frame of erosion of a stent.

In general, it is desirable for the bioceramic particles to be dispersed with high uniformity throughout the polymeric matrix. A more uniform the dispersion of the particles results in more uniform properties of the composite and a device fabricated from the composite. For example, a uniform dispersion can result in a greater uniformity in the increase in toughness, modulus, strength, and degradation rate.

Further embodiments of the invention include formation of the bioceramic-polymeric matrix composite and fabrication of an implantable medical device, such as a stent, therefore, in some embodiments, the polymer matrix composite can be formed by mixing the polymeric matrix with the bioceramic particles and extruding the mixture to form a construct, such as a tube. A stent can then be fabricated from the tube. For example, a stent pattern can then cut into the tube by laser cutting.

The mixing or extrusion process can be performed at low temperature, such as near room temperature (20-30.degree. C.) or slightly elevated temperatures (<50.degree C. above room temperature). In other embodiments, the mixing or extrusion process can be performed at high temperature, for example, 50-75% of the melting temperature of the polymer. In some embodiments, the mixing or extrusion are performed at temperatures above 75% of the melting temperature of the polymer or greater than the melting temperature of the polymer. The mixing or forming is performed at a temperature below the melting point of the bioceramic particles. The temperature can also be below a temperature at which the bioceramic particles significantly chemically degraded.

In some embodiments, the mixing and forming can be performed in the same apparatus. In such embodiments, the polymeric particles and bioceramic particles can be fed into a mixing apparatus, such as an extruder, which both mixes and forms the construct. Alternatively, the composite mixture of polymer and bioceramic particles can be mixed separately in one apparatus. For example, the composite can be formed in an extruder or batch mixer. In one embodiment, the bioceramic particles can be combined with a polymer in a powdered or granular form prior to the mixing of the particles with the polymer at an elevated temperature. The formed composite can then be fed into an extruder to form the tube.

Agglomeration or formation of clusters of bioceramic particles can reduce the uniformity of dispersion of the particles in the polymer matrix. The agglomeration of bioceramic particles makes it difficult to disperse the particles within the composite. The presence of larger clusters in the composite tends to result in a decrease in material performance. Such larger clusters can result in the formation of voids in a composite portion of a stent, which are preferential sites for crack initiation and failure. The mechanical mixing in a conventional single screw extruder or in batch processing can be insufficient to break up the clusters, resulting in a nonuniform mixture of bioceramic particles and polymer.

Various methods may be employed to increase the uniformity of dispersion of bioceramic particles within a polymer matrix. Certain embodiments for decreasing agglomeration and increasing the dispersion of bioceramic particles in a composite can include processing a mixture of particles and polymer with mechanical methods sufficient to reduce agglomeration. Such embodiments can include processing, a mixture of a polymer and agglomerated bioceramic particles under high shear stress conditions. Some embodiments can include processing the mixture such that the particles are subjected to shear stress higher than the fracture strength of the agglomerated particles. In one embodiment, a polymer can blended or mixed with bioceramic particles in a manner that subjects the mixture to a shear stress higher than the fracture strength of agglomerates of bioceramic particles. Thus, polymer-bioceramic particle mixture can be processed so that a maximum shear stress generated during mixing is higher than the fracture strength of the bioceramic particle agglomerates. Agglomerated particles may be mechanically broken down and more uniformly dispersed within the polymer.

It is believed that the shear stress produced by a single screw extruder is typically lower than the fracture strength of bioceramic particle agglomerates. Various kinds of mixing devices may be employed that can apply a shear stress higher than the fracture strength of agglomerates. Mechanical blending devices that can apply a sufficiently high shear stress include, but are not limited to, a twin screw extruder or kneader. During blending, once the shear stress is higher than the fracture strength of bioceramic particles agglomerates, the agglomerates are broken down and more uniformly dispersed into the polymer. The polymer and bioceramic particles can be fed into a mechanical blending device separately and processed at high shear stress. Alternatively, a composite mixture of polymer and bioceramic particles can be fed into a mechanical blending device and processed at the high shear stress.

The bioceramic particle/polymer mixture can be processed at the sufficiently high shear stress for a time sufficient to reduce agglomeration and disperse the particles. For example, the mixture can be processed between about 5 min. to about 30 min., more narrowly about 8 min. to about 20 min., or more narrowly about 10 min to about 15 min. In one embodiment, the composite formed with surface modified bioceramic particles can also be processed in this manner.

In general, good bonding between a matrix and a discrete or reinforcing phase in a composite material facilitates improvement of the mechanical performance of the composite. For example, the increase in the strength and toughness of composite due to the bioceramic particles can be enhanced by good bonding between the polymer matrix and particles.

In some embodiments, bioceramic particles may include an adhesion promoter to improve the adhesion between the particles and the polymer matrix. In one embodiment, an adhesion promoter can include a coupling agent. A coupling agent refers to a chemical substance capable of reacting with both the bioceramic particle and the polymer matrix of the composite material. A coupling agent acts as an interface between the polymer and the bioceramic particle to form a chemical bridge between the two to enhance adhesion.

The adhesion promoter may include, but is not limited, to, silane and non-silane coupling agents. For example, the adhesion promoter may include 3-aminopropyltrimetboxysilane, 3-aminopropyltriethoxysilane, aminopropylmethyldiethoxy silane, organotrialkoxysilanes, titanates, zirconates, and organic acid-chromium chloride coordination complexes.

In some embodiments, the surface of the bioceramic particles may be treated with at adhesion promoter prior to mixing with the polymer matrix. In one embodiment, the bioceramic particles can be treated with a solution containing the adhesion promoter. Treating can include, but is not limited to, coating, dipping, or spraying the particles with an adhesion promoter or a solution including the adhesion promoter. The particles can also be treated with a gas containing the adhesion promoter. In one embodiment, treatment of the bioceramic particles includes mixing the adhesion promoter with solution of distilled water and a solvent such as ethanol and then adding bioceramic particles. The bioceramic particles can then be separated from the solution, for example, by a centrifuge, and the particles can be dried. The bioceramic particles may then used to form a composite. In an alternative embodiment, the adhesion promoter can be added to the particles during formation of the composite. For example, the adhesion promoter can be mixed with a bioceramic/polymer mixture during extrusion.

### EXAMPLES

The example set forth below is for illustrative purposes only and are in no way meant to limit the invention. The following example is given to aid in understanding the invention, but it is to be understood that the invention is not limited to the particular materials or procedures of examples.

### Example 1

### PLLA/ACP Composite Tube Extrusion

ACP (Sigma-Aldrich. Size<150 nm; Ca/P˜1:1) was uniformly dispersed into PLLA (Mᵥ=250,000, Purac Biomaterials, Lincolnshire, Ill. USA) with a ratio of PLLA/ACP of 98/2 (w/w), using a speed mixer under class 100 clean room conditions. 2% paclitaxel (w/w) (Sigma-Aldrich) was incorporated into both PLLA and PLLA/ACP composites prior to extrusion in a single screw extruder (Genca Engineering Inc., Saint Petersberg Fla.). The final PLLA and PLLA/ACP tube dimensions were 1.8 mm OD×150 μm wall thickness.

### Examples 2

### Invented Stent Fabrication

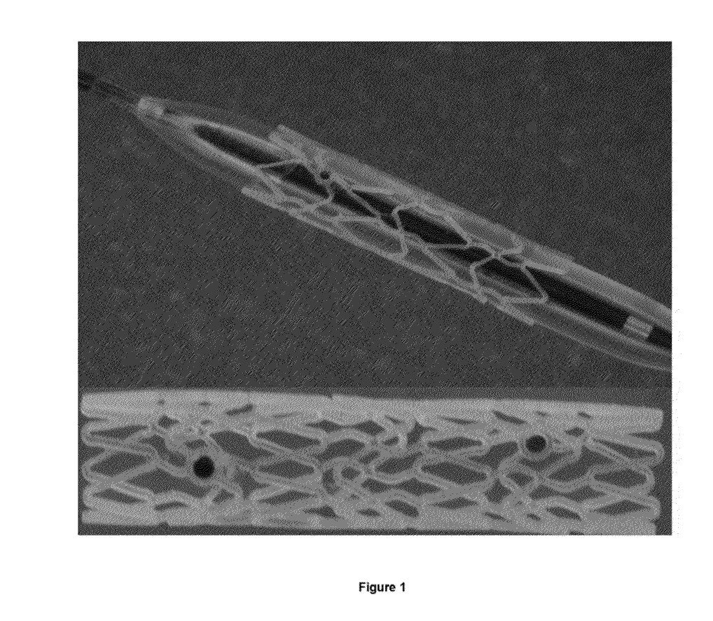

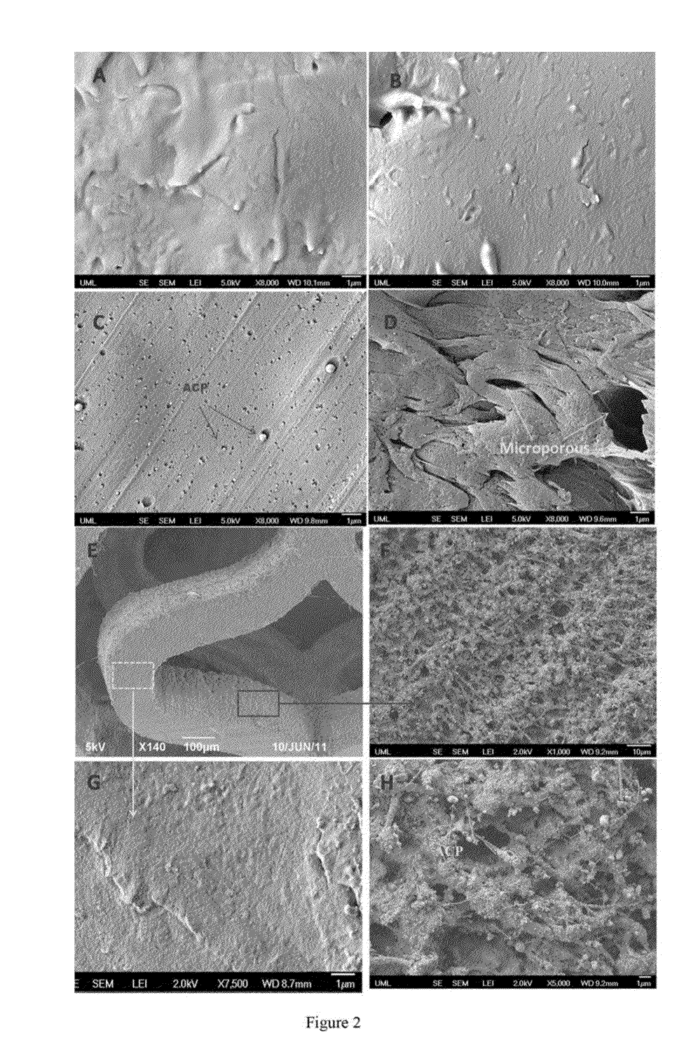

Tubes extruded from Example 1 study were cut from a femtosecond laser according to design specification. The stent strut thickness is 1.50 um which is the same as the tube thickness. FIG. 1 shows the images of invented PLLA/ACP stent. FIG. 2E shows the precisely cut stent strut.

### Examples 3

### Mechanic Property Measurement of Tube Extruded from PLLA/ACP Composite

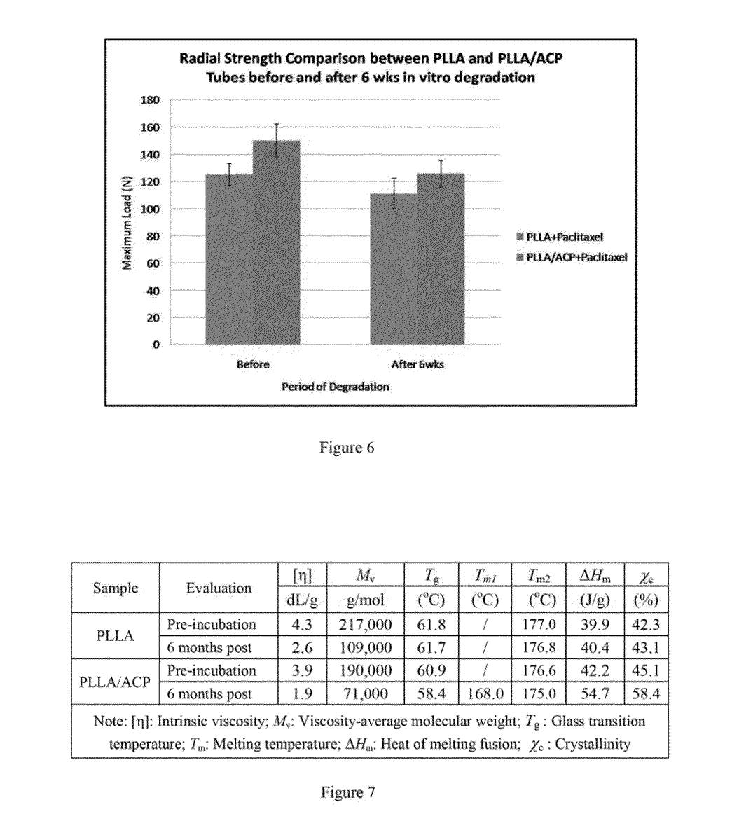

The radial strength of PLLA and PLLA/ACP tubes (1.3 mm) before and after a 6 week degradation period were evaluated using a catheter tensile testing machine (Model 4400R, Instron Inc., Norwood, Mass.) with a flat-plate compression testing rate at 0.05 in/min. The maximum load-at-crush from each tube was recorded. Data shows that pressures required to collapse PLLA/ACP tubes were significantly greater than PLLA tubes before (150.4±12.1N vs. 1.25.3±8.2N, P<0.01) and after 6-week degradation (125.9±9.8N vs 111.3±11.0N, P<0.01) (FIG. 6). Moreover, pressures for the PLLA/ACP composite tube after degradation were virtually identical for PLLA tubes before degradation (125.9±9.8N vs. 125.3±8.2N, respectively). Both PLLA/ACP and PLLA tubes showed comparable and modest decreases in radial strength after 6 weeks degradation (15.60±11.25% vs. 10.70±11.7%, respectively, P>0.05) suggesting that ACP enhanced radial strength for the dimension of PLLA/ACP tube tested.

### Example 4

### Structure Characterization of PLLA/ACP Composite

Scanning images were taken of razor-cut cross-sections of PLLA (FIGS. 2A & 2B) and PLLA/ACP composite tubes (FIGS. 2C & 2D) before and after 6 weeks hydrolytic degradation. PLLA tubes exhibited a relatively smooth cross-section surface pre-degradation (FIG. 2A), while, the crossing area of PLLA/ACP composite tube showed a uniform dispersion of ACP particles embedded within the PLLA matrix (FIG. 2C) with porous structure formed around these particles. After 6 weeks degradation, no gross structural changes were observed with the PLLA tube (FIG. 2B); however, pore size increased and became irregular for the PLLA/ACP composite tubes. These observations indicate an accelerated degradation of PLLA in the presence of ACP. The cross-sectional surface of a PowerStent® Absorb stent cut with ultrafast femtosecond laser is shown in FIG. 2E-2H. The stent was cut precisely with no thermal damage evident along strut edges (FIG. 2E) and along its smooth surface post extrusion (FIG. 2G). A uniformly microporous surface is created by laser processing, unlike the razor-cut tube surface. It is possible that ACP particles may become dissociated on the laser-cut surface during processing.

### Example 5

### In-Vitro Degradation of PLLA/ACP Composite

Twelve equivalent tube segments of PLLA and PLLA/ACP composite materials were incubated individually in sealed containers of saline submerged in a 37° C. water bath (Precision Scientific Inc., Chicago, Ill.) and shaken constantly at 30 rpm. Saline solutions was replaced daily to keep the degradation medium fresh. Samples from each group were collected after 6 weeks and 6 months and dried at room temperature for 48 hours prior to performance testing. The molecular weight (MW) changes of both PLLA and PLLA/ACP after 6 months degradation period were evaluated using an Ubbelholde Viscometer. PLLA and PLLA/ACP tubes were dissolved in chloroform. The elution time of the pure chloroform solvent was determined and used as a reference standard. Five different concentrations of PLLA and PLLA/ACP solutions were prepared. The elution time from each concentration of the sample solutions was recorded. The relative viscosity for each concentration was obtained with respect to the pure solvent. The viscosity-average molecular weight was calculated with Mark-Houwink equation; [η]KMᵥ^{α}, Where K=5.45×10⁻⁴ and α=0.73. FIG. 7 summarized the physical properties of PLLA and PLLA/ACP composites tubes before and after 6 month hydrolytic degradation. The average MW of the raw PLLA material was determined approximately 250,000 g/mol. The extrusion processing reduced PLLA molecular weight substantially for PLLA (12.4%; 217,000 g/mol) and PLLA/ACP (24%; 190,0000 g/mol) following thermal extrusion. After 6 months degradation, the viscosity-average molecular weight (Mᵥ) of neat PLLA decreased 49.8% from 217,000 g/mol to 109,000 g/mol; while the Mᵥ of PLLA/ACP decreased 62.6% composite from 119,0000 g/mol to 71,000 g/mol. These data indicate that ACP accelerated the hydrolytic degradation of PLLA.

### Example 6

### In-Vivo Performance of PLLA/ACP Stent

to further investigate the in-vivo performance of invented PLLA/ACP stent, 6 PLLA and 6 PLLA/ACP stents were implanted into 12 domestic mini pig coronary arteries. Thirty days post implantation, animals were euthanized, and the stented arteries were analyzed angiographically and pathologically.

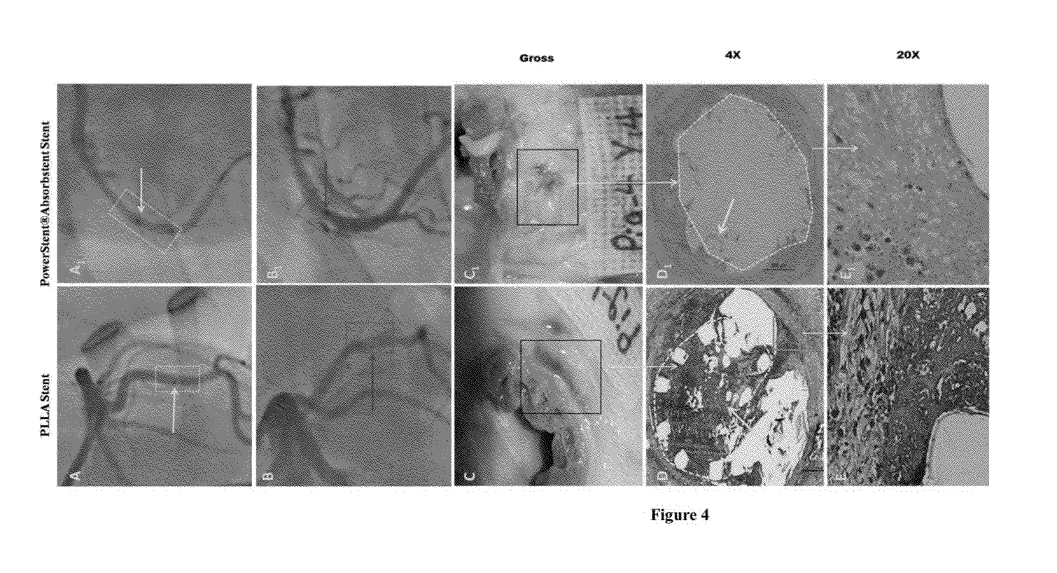

Angiography performed immediately post-deployment showed no evidence vascular injury once fully-expanded to 3.0 mm (FIGS. 4A and 4A₁). Histology showed all arterial samples from the PLLA group show evidence of recoil and restenosis (FIG. 4B) while only one in the PowerStent® Absorb group showed similar changes. Angiography for that subject revealed contrast media leakage into the surrounding myocardium. A displaced metal radiopaque marker was later confirmed as the cause for the leakage and pathology (data not shown).

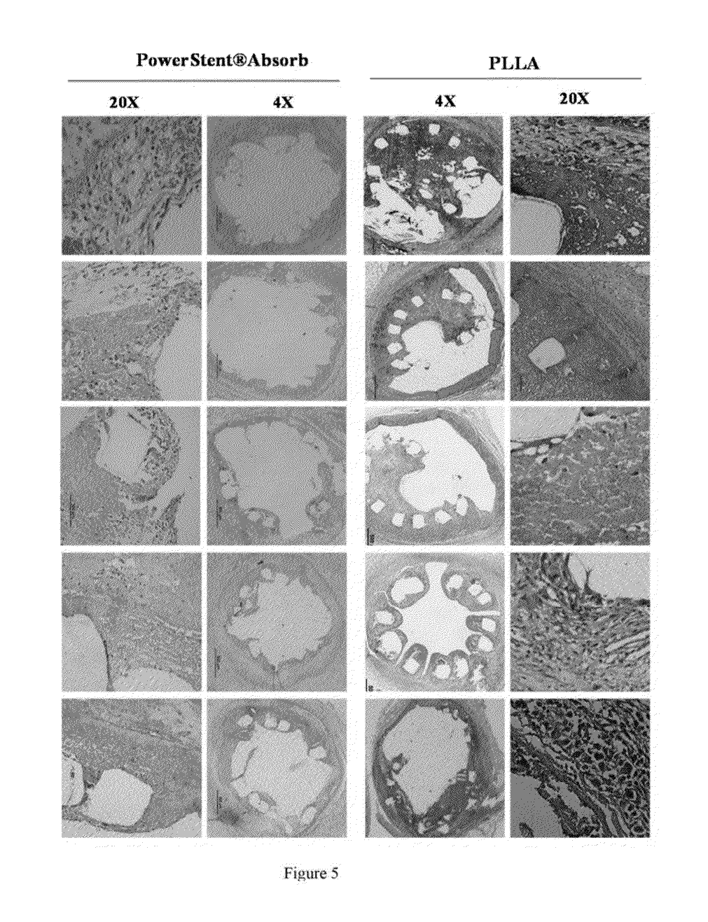

At necropsy, stented coronary arteries segments were dissected to reveal presence of inflammation. Extensive inflammation was evident in 4 of 5 PLLA-stented coronary samples (FIG. 4C). One subject in the PowerStent® Absorb stent group with similar inflammation was previously described with a dislocated marker. No other subjects in that group showed notable pathological changes. The tissue inflammation can only be found after removing the fat tissue surrounding the PowerStent® Absorb stented arteries (FIG. 4C₁). A case-by-case comparison of represented histopathology is summarized for both treatment groups in FIG. 5.

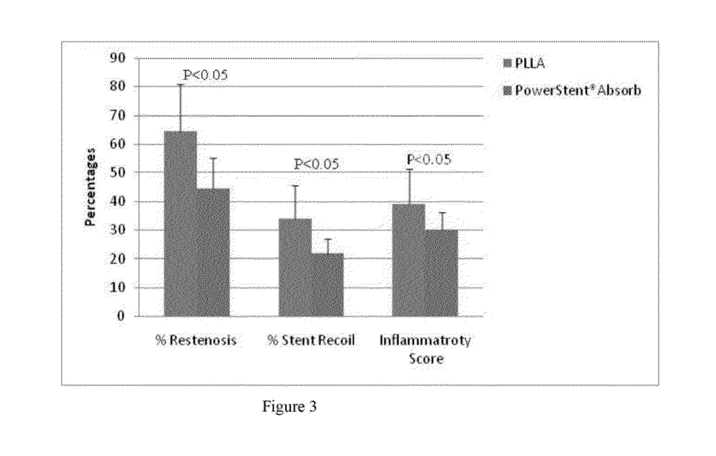

Morphometric analyses revealed treatment related difference in stenosis, stent recoil, and inflammatory cell infiltration between PLLA-stented and PowerStent® Absorb stented arteries. FIG. 3 shows evidence of significantly greater stenosis in the PLLA-stented group relative to the PowerStent® Absorb group (64.47±16.2% vs. 44.49±10.59. P<0.05), greater stent recoil (33.81±11.49% vs. 21.57±5.36, P<0.05), and a higher inflammatory score (38.9±12.1 vs. 30.1±6.1, P<0.01). No evidence of thrombus was observed in any of the survivors although, 4 out of 5 stented arteries in PLLA group showed massive neointima formation and inflammatory cell infiltration (FIGS. 4D&E and FIG. 5). Excluding one marker-injured artery, all other 5 subjects in the PowerStent® Absorb group show a thin layer of presumed endothelial cells (FIG. 4D₁ yellow arrow) covering each exposed strut. In contrast to all PLLA-stented samples showing evidence of vessel recoil (FIG. 4D, dashed yellow circle, and FIG. 5), PowerStent® Absorb group presented the absence of vessel recoil (FIG. 4D₁ dashed yellow circle, and FIG. 5).

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A biodegradable stent comprising a stent body formed from a polymer-bioceramic nanoparticle matrix composite. The composite include bioerodible bioceramic particles dispersed within a biodegradable polyester polymer, wherein the dispersed bioceramic particles modify the degradation rate, reinforce the mechanical properties and improve the biocompatibility of the stent body in a vascular environment.

2. The stent of claim 1, wherein said biodegradable polyester polymer is selected from the group consisting of Poly(D,L-lactide-co-glycolide) (PLGA), polylactides (PLA), Poly(L-lactide) (PLLA), Poly(D,L-lactide) (PDLA), polyglycolides (PGA), or combination thereof.

3. The stent of claim 1, wherein the said particles are selected from the group consisting of Amorphous Calcium Phosphate (ACP), Dicalcium Phosphate (DCP), Tricalcium Phosphate (α-TCP), Tricalcium Phosphate (β-TCP), Pentacalcium Hydroxyl Apatite (HA), and Tetracalcium Phosphate Monoxide (TTCP), etc. or combination thereof.

4. The stent of claim 1, wherein the composite comprises 1 wt % to 50 wt % of the bioceramic particles.

5. The stent of claim 1, wherein the erosion of the particles increases the erosion rate of the polymer, thereby decrease the time for the stent to completely absorb.

6. The stent of claim 1, wherein the particles have basic degradation products that neutralize the acidic degradation environment for the polymer, thereby inhibit the tissue inflammatory formation.

7. The stent of claim 1, wherein a time for the stent to completely absorb is greater than six months.

8. The stent of claim 1, wherein the bioceramic particles increase the tensile strength of the composite.

9. The stent of claim 1, wherein the bioceramic particles improve the biocompatibility of the composite.

10. The stent of claim 1, wherein the stent body was encapsulated with at least one therapeutic agent that eluted over time.

11. The stent of claim 10, wherein said therapeutic agent is selected from the group consisting of anti-neoplastic and immunosuppressive agent.

12. The stern of claim 11, wherein said anti-neoplastic agent is selected from the group consisting of paclitaxel, carboplatin. vinorelbine, doxorubicin, gemcitabine, actinomycin-D, cisplatin, camptothecin, 5-fluorouracil, cyclophosphamide, 1-β-D-arabinofuranosylcytosine, and combinations or analogs thereof.

13. The stent of claim 11, wherein said immunosuppressive agent is selected from the group consisting of sirolimus, zotarolimus, tacrolimus, everolimus, biolimus, pimecrolimus, supralimus, temsirolimus, TAFA 93, invamycin and neuroimmunophilins, and combinations or analogs thereof.

14. A method of making a biodegradable stent comprising: processing bioceramic particles with an biodegradable polymer to form a composite, wherein the polymer and the particles are processed with a shear stress higher than the fracture strength of clusters of agglomerated bioceramic particles so that agglomeration of the particles is reduced; forming a tube from the composite; and fabricating a stent from the tube, wherein the dispersed bioceramic particles modify the degradation rate, enhance mechanical properties, and improve the biocompatibility of the stent body in a vascular environment.

15. The method of claim 14, wherein processing the bioceramic particles with the biodegradable polymer comprises mixing the bioceramic particles and the polymer in a twin-screw extruder or a kneader in such a way that agglomeration is reduced.

16. The method of claim 14, wherein the bioceramic particles are nanoparticles.

17. The method of claim 16, wherein said nanoparticles are selected from the group consisting of Amorphous Calcium Phosphate (ACP), Diealcium Phosphate (DCP), Tricalcium Phosphate (α-TCP), Tricalcium Phosphate(β-TCP), Pentacalcium Hydroxyl Apatite(HA), and Tetracalcium Phosphate Monoxide(TTCP), etc. or combination thereof.
